(19) United States
(12) Patent Application Publication
(10) Pub. No.: US 2019/0218498 A1
(43) Pub. Date: Jul. 18, 2019

(54) DOUBLE-CIRCULATION TEMPERATURE-CONTROLLED CELL INCUBATOR AND WORKING METHOD THEREOF

(71) Applicants: CHINA INNOVATION INSTRUMENT CO., LTD., Ningbo (CN); NINGBO UNIVERSITY, Ningbo (CN)

(72) Inventors: Luhong WEN, Ningbo (CN); Shundi HU, Ningbo (CN); Zhenzhi SHI, Ningbo (CN); Jianqin GAN, Ningbo (CN); Lulu SHI, Ningbo (CN)

(21) Appl. No.: 16/288,017

(22) Filed: Feb. 27, 2019

Related U.S. Application Data
(63) Continuation of application No. PCT/CN2017/110249, filed on Nov. 9, 2017.

(30) Foreign Application Priority Data
Nov. 10, 2016 (CN) 201610988186.5

(51) Int. Cl. C12M 1/00; C12M 1/02

(52) U.S. Cl. CPC C12M 41/14; C12M 41/20

(57) ABSTRACT

A double-circulation temperature-controlled cell incubator and a working method thereof. The cell incubator includes a first incubator body, a second incubator body arranged inside the first incubator body, an enclosed space formed between the first incubator body and the second body, a third incubator body arranged inside the second incubator body, a transport device, a heating device, and an insulation device arranged on an outer side or inner side of the first incubator body. An upper side, a rear side and a bottom side of the third incubator body and an inner wall of the second incubator body form a space. The transport device is configured to transport gas in the third incubator body to the space. The heating device is arranged at an inner side of a door or a side of the second incubator body.

### CROSS-REFERENCE TO RELATED APPLICATIONS

This application is a continuation of International Patent Application No. PCT/CN2017/110249, filed on Nov. 9, 2017, which claims the benefit of priority from Chinese Application No. 201610988186.5, titled “DOUBLE-CIRCULATION TEMPERATURE CONTROL CELL INCUBATOR AND WORKING METHOD THEREOF”, filed on Nov. 10, 2016. The contents of the aforementioned applications, including any intervening amendments thereto, are incorporated herein by reference.

### TECHNICAL FIELD

The present application relates to cell culture, and more particularly to a double-circulation temperature-controlled cell incubator and a working method thereof.

### BACKGROUND

Incubator is a device for culturing various types of cells and microorganisms such as viruses under the simulated in vivo physiological environment. Incubators typically have excellent characteristics of high uniformity, good stability and rapid recovery of gas concentration, temperature and humidity, no condensation of water, etc. To achieve these characteristics, the existing incubators mainly include the following types.

1. Turbulent-Flow Incubator.

This incubator uses a fan to stir the gas therein to flow, which leads to significant local convection and sufficient heat exchange, but non-uniform global heat exchange and vortex and local blind areas at the same time, easily causing bacterial growth.

2. Laminar-Flow Incubator.

This mode forms a laminar flow in the incubator. The temperature inside the incubator is relatively uniform, but the temperature outside the incubator is non-uniform. There is a significant difference between the inside and the outside temperatures; especially when the incubator is suspended or shut down, condensate water is likely to occur on the inner wall, door and multi-path tube wall.

3. Air-Jacketed Laminar-Flow Incubator.

In this incubator, a hot gas flow around the incubator is formed to provide good temperature uniformity. However, the heating speed and efficiency are not desirable.

The above modes affect the culture time more or less, prolonging the whole cycle of cell culture. The growth of bacteria may be promoted, thereby affecting the success rate and quality of cell culture. In view of some defects of the existing heating devices, there is a need to develop an incubator equipped with a heating device to provide high control precision of gas concentration, temperature and humidity, good uniformity and rapid recovery without condensate water.

### SUMMARY OF THE INVENTION

The present invention provides a double-circulation temperature-controlled cell incubator with accurate temperature control and good temperature uniformity to overcome the above defects in the prior art.

An object of the present invention is to provide a double-circulation temperature-controlled cell incubator, including:
a first incubator body;
a second incubator body arranged inside the first incubator body, wherein an enclosed space is formed between the first incubator body and the second incubator body;
a third incubator body arranged in the second incubator body, wherein a distance between an upper side of the third incubator body and an inner wall of the second incubator body, a distance between a rear side of the third incubator body and the inner wall of the second incubator body and a distance between a bottom side of the third incubator body and the inner wall of the second incubator body are each greater than zero to form a space that is in communication with an outside through a gas inlet; an interior of the third incubator body is in communication with the space via through holes provided on the upper side and the bottom side of the third incubator body;
a door arranged at a front side of the first incubator body, wherein a sealing member is arranged between the first incubator body and the door;
a heating device arranged at an inner side of the door, at a side of the second incubator body or in the enclosed space;
a transport device configured to feed gas in the third incubator body into the space and transport gas at a lower portion of the enclosed space to an upper portion of the enclosed space; and
an insulation device arranged on an outer side of the second incubator body or on an inner side of the first incubator body.

Another object of the present invention is to provide a working method of the double-circulation temperature-controlled cell incubator, comprising the following steps:
turning on the heating device and the transport device to transport gas in the third incubator body to the space between the upper side of the third incubator body and the second incubator body to mix with gas entering from the gas inlet; allowing the mixed gas to flow downward along a space between the rear side of the third incubator body and the second incubator body to a space between the bottom side of the third incubator body and the second incubator body and finally to enter the third incubator body;
heating a gas between the first incubator body and the second incubator body by the heating device; and transporting the heated gas to flow upward along a space between the rear side of the second incubator body and the first incubator body to a space between the upper side of the second incubator body and the first incubator body, and then flow downward along a space between a left side and/or right side of the second incubator body and the first incubator body to a space between the bottom side of the second incubator body and the first incubator body.

The present invention has the following beneficial effects as compared to the prior art.

1. Fast heating rate. The heating device(s) and the transport device(s) are provided in the space between the first incubator body and the second incubator body, allowing for a higher environmental temperature inside the third incubator body. Therefore, the heating rate and the temperature recovery rate of the cell culture chamber are accelerated.

2. High-accuracy temperature control, good temperature uniformity and high reliability. The double-circulation mode can improve the temperature uniformity of the cell culture chamber in the third incubator body, thus increasing the precision of temperature control. Additionally, the double-circulation heating avoids the failure of the entire equipment caused by the failure of some heating members.

3. No condensate water. Additional heating device(s) and transport device(s) are provided in the enclosed space, such that the closed space is maintained at a temperature consistent with the culture chamber in the third incubator body, thereby effectively solving the problem of water vapor condensation on the sensor and various bypass systems.

### BRIEF DESCRIPTION OF THE DRAWINGS

The disclosure of the present invention will become more apparent with reference to the accompanying drawings. It should be understood by those skilled in the art that these drawings are merely illustrative of the present invention without limiting the scope of the present invention.

FIG. 1 is a schematic sectional view of a double-circulation temperature-controlled cell incubator according to an embodiment of the present invention.

### DETAILED DESCRIPTION OF EMBODIMENTS

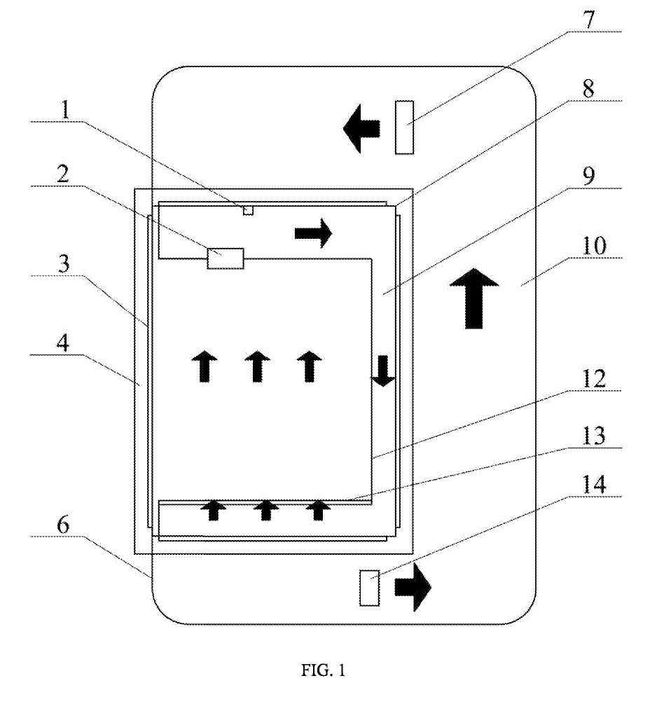

FIG. 1 and the following description describe optional embodiments of the present invention to teach those skilled in the art how to implement and reproduce the present invention. Some conventional aspects have been simplified or omitted so that the present invention becomes more apparent. It should be understood by those skilled in the art that variants or substitutions derived from these embodiments shall fall within the scope of the present invention. It should be understood by those skilled in the art that the following features can be combined in various ways to form various variants of the present invention. Therefore, the present invention is not limited to the following optional embodiments, but is only defined by the appended claims and equivalents thereof.

### EXAMPLE 1

FIG. 1 schematically shows a sectional view of a double-circulation temperature-controlled cell incubator according to an embodiment of the present invention. As shown in FIG. 1, the double-circulation temperature-controlled cell incubator includes a first incubator body 6, a door 4, a second incubator body 8, a third incubator body 12, a plurality of heating devices, three transport devices 2, 7 and 14, and an insulation device.

The first incubator body 6 is made from an electropolished stainless steel material.

The door 4 is arranged on a front side of the first incubator body 6. A sealing member is provided between the first incubator body 6 and the door 4.

The second incubator body 8 is arranged inside the first incubator body 6, and an enclosed space 10 is formed between the first incubator body 6 and the second incubator body 8.

The third incubator body 12 is arranged in the second incubator body 8. A distance between an upper side of the third incubator body and an inner wall of the second incubator body, a distance between a rear side of the third incubator body and the inner wall of the second incubator body and a distance between a bottom side of the third incubator body and the inner wall of the second incubator body are each greater than zero to form a space 9 that is in communication with an outside through a gas inlet. An interior of the third incubator body is in communication with the space 9 via through holes provided on the upper side and the bottom side of the third incubator body 12.

The heating devices are arranged at an inner side of the door 4, at a side of the second incubator body 8 and/or in the enclosed space 10. Specifically, the heating devices consists of six heating sheets 3 and two heaters. The six heating sheets 3 are respectively arranged at the inner side of the door 4 and at an upper side, a bottom side, a rear side, a right side and a left side of the second incubator body 8. The two heaters are respectively arranged between the upper side of the second incubator body 8 and the first incubator body 6 and between a lower side of the second incubator body 8 and the first incubator body 6.

The three transport devices 2, 7 and 14 are configured to transport a gas in the third incubator body into the space 9 and transport a gas at an inner bottom of the enclosed space 10 to an upper portion of the enclosed space 10, where the enclosed space 10 is formed between the second incubator body 8 and the first incubator body 6.

The insulation device is arranged on an inner wall of the first incubator body 6 or on an outer wall of the second incubator body 8 to form an insulation enclosed space between the first incubator body 6 and the second incubator body 8.

This embodiment of the present invention further provides a working method of the double-circulation temperature-controlled cell incubator, including the following steps.

The heating devices and the transport devices are turned on, such that the gas in the third incubator body 12 is fed to a space between the upper side of the third incubator body 12 and the second incubator body, and then mixes with a gas entering through the gas inlet 1. The mixed gas flows downward along a space between the rear side of the third incubator body 12 and the second incubator body 8 to a space between the bottom side of the third incubator body 12 and the second incubator body 8 and finally enters the third incubator body 12.

A gas between the first incubator body 6 and the second incubator body 8 is heated with the heating devices. The heated gas is transported by the transport devices to flow upward along a space between the rear side of the second incubator body 8 and the first incubator body 6 to a space between the upper side of the second incubator body 8 and the first incubator body 6, and then to flow downward along a space between a left side of the second incubator body 8 and the first incubator body 6 and/or a space between a right side of the second incubator body 8 and the first incubator body 6 to a space between the bottom side of the second incubator body 8 and the first incubator body 6.

### EXAMPLE 2

An application of the double-circulation temperature-controlled cell incubator in Example 1 of the present invention is provided.

As shown in FIG. 1, in this application, three fans are used as the transport devices. A first fan 2 is arranged at the upper side of the third incubator body 12. A second fan 7 is arranged in a space between the upper side of the second incubator body 8 and the first incubator body 6. A third fan 14 is arranged in a space between the bottom side of the second incubator body 8 and the first incubator body 6. Eight heating devices consisting of six heating sheets and two heaters are employed. The six heating sheets are respectively arranged on the inner side of the door 4 and outer walls of the upper side, the bottom side, the rear side, the right side and the left side of the second incubator body 8. The other two heaters combine with the fan in the enclosed space 10 to form a fan heater. The insulation device is made from an insulation material and is arranged on the inner wall of the first incubator body 6. The bottom side of the third incubator body 12 is a perforated plate 13 to allow gas to enter the third incubator body 12.

A working method of the cell incubator in this embodiment of the present invention includes the following steps.

The heating devices and the transport devices are turned on. The gas in the third incubator body 12 (i.e., the cell culture chamber) is allowed to flow upward under the pumping of the fan on the upper side of the third incubator body 12, and then mixes with a newly entered gas. The mixed gas flows downward along a space between the third incubator body 12 and the second incubator body 8 and finally enters the third incubator body 12 through the perforated plate 13, such that a gas circulation is completed to improve the temperature uniformity.

The gas at the inner bottom of the enclosed space formed by the first incubator body 6 and the second incubator body 8 is heated and pumped to enter a space between the rear side portion of the second incubator body 8 and the first incubator body 6. The heated gas flows upward and enters a space between the upper side of the second incubator body 8 and the first incubator body 6. The gas is heated and pumped again with the fan heater to flow downward along a space between the right side of the second incubator body 8 and the first incubator body 6 and a space between the left side of the second incubator body 8 and the first incubator body 6, such that a gas circulation in the enclosed space is completed.

1. A double-circulation temperature-controlled cell incubator, comprising:
a first incubator body;
a second incubator body arranged inside the first incubator body, wherein an enclosed space is formed between the first incubator body and the second incubator body;
a third incubator body arranged in the second incubator body, wherein a distance between an upper side of the third incubator body and an inner wall of the second incubator body, a distance between a rear side of the third incubator body and the inner wall of the second incubator body and a distance between a bottom side of the third incubator body and the inner wall of the second incubator body are each greater than zero to form a space that is in communication with an outside through a gas inlet; an interior of the third incubator body is in communication with the space via through holes provided on the upper side and the bottom side of the third incubator body;
a door arranged at a front side of the first incubator body, wherein a sealing member is arranged between the first incubator body and the door;
a heating device arranged at an inner side of the door, at a side of the second incubator body or in the enclosed space;
a transport device configured to feed gas in the third incubator body into the space and transport a gas at a bottom portion of the enclosed space to an upper portion of the enclosed space; and
an insulation device arranged on an outer side of the second incubator body or on an inner side of the first incubator body.

2. The cell incubator of claim 1, wherein the heating device is arranged on an outer wall of the second incubator body.

3. The cell incubator of claim 1, wherein in the enclosed space, the heating device is arranged between an upper side of the second incubator body and a top of the first incubator body, and/or arranged between a lower side of the second incubator body and a bottom of the first incubator body.

4. The cell incubator of claim 1, wherein the transport device is arranged between an upper side of the second incubator body and a top of the first incubator body, and/or arranged between a lower side of the second incubator body and a bottom of the first incubator body.

5. The cell incubator of claim 1, wherein the transport device is arranged on the upper side of the third incubator body.

6. The cell incubator of claim 1, wherein the transport device is a fan.

7. The cell incubator of claim 1, wherein the transport device and the heating device in the enclosed space are combined to form a fan heater.

8. The cell incubator of claim 1, wherein the insulation device is arranged on an inner wall of the first incubator body or on an outer wall of the second incubator body.

9. The cell incubator of claim 1, wherein the insulation device is made from an insulation material.

10. The cell incubator of claim 1, wherein a bottom of the third incubator body is a perforated plate.

11. The cell incubator of claim 1, wherein a cross section between a rear side wall of the second incubator body and a rear side wall of the first incubator body is in a shape of a cross section of venturi.

12. The cell incubator of claim 1, wherein a cross section between a rear side wall of the third incubator body and a rear side wall of the second incubator body in a shape of a cross section of venturi.

13. The cell incubator of claim 1, wherein the third incubator body is provided with a temperature measuring instrument configured to sense temperature inside the third incubator body.

14. The cell incubator of claim 1, wherein the gas inlet is connected to a preheating tube and an external gas enters the space from the gas inlet through the preheating tube.

15. A working method of the cell incubator of claim 1, comprising:
turning on the heating device and the transport device to transport gas in the third incubator body to the space between the upper side of the third incubator body and the second incubator body to mix with gas entering from the gas inlet; allowing the mixed gas to flow downward along a space between the rear side of the third incubator body and the second incubator body to a space between the bottom side of the third incubator body and the second incubator body and finally to enter the third incubator body;
heating gas between the first incubator body and the second incubator body by the heating device; and transporting the heated gas to flow upward along a space between the rear side of the second incubator body and the first incubator body to a space between the upper side of the second incubator body and the first incubator body, and then to flow downward along a space between a left side and/or right side of the second incubator body and the first incubator body to a space between the bottom side of the second incubator body and the first incubator body.
